(19) United States
(12) Patent Application Publication
(10) Pub. No.: US 2010/0056899 A1
(43) Pub. Date: Mar. 4, 2010

(54) METHOD AND APPARATUS FOR MULTIMODAL IMAGING

(75) Inventors: Steve Toddes, Westminister, MA (US); Craig Ferris, Holden, MA (US); Rostislav Lemdiasov, Holden, MA (US)

Correspondence Address: Leason Ellis LLP, 81 Main Street, Suite 503, White Plains, NY, 10601, US

(73) Assignee: EKAM Imaging Inc., SHREWSBURY, MA (US)

(21) Appl. No.: 12/546,977

(22) Filed: Aug. 25, 2009

Related U.S. Application Data
(60) Provisional application No. 61/183,344, filed on Jun. 2, 2009.
(60) Provisional application No. 61/092,116, filed on Aug. 27, 2008.

(51) Int. Cl. A61B 5/055; A01K 15/04

(52) U.S. Cl. 600/411; 119/729

(57) ABSTRACT

The apparatus and imaging method of the present invention incorporates and permits the use of a single technology to combine MR, SPECT and PET in awake animal imaging studies.

### CROSS REFERENCE TO RELATED APPLICATIONS

The present application claims the benefit of U.S. patent application Ser. No. 61/183,344, filed Jun. 2, 2009 and U.S. patent application Ser. No. 61/092,116, filed Aug. 27, 2008, which are hereby incorporated by reference in their entirety.

### TECHNICAL FIELD

The present invention relates to imaging and more particularly, relates to an apparatus and method for multimodal imaging in conscious animals.

### BACKGROUND

Human studies utilizing functional magnetic resonance imaging (fMRI) have advanced our understanding of the regional and functional interplay between populations of neurons serving sensory, integrative and motor functions. While most work on fMRI has been done in humans, it has been difficult to use this technology in conscious animals because of motion artifact. As a result, most studies to date have been limited to animals which are typically anesthetized in order to minimize this problem of motion artifacts. The low level of arousal during anesthesia either partially or completely suppresses the fMRI response and has impeded fMRI application to the more physiologically relevant functions that have been noted in humans.

There are a number of different imaging techniques available for imaging an object, such as a person or conscious animal, in order to gain helpful information about a local area of interest, such as the brain or an organ, etc.

For example, one type of imaging technique is known as single photon emission computed tomography (SPECT) which is a nuclear medicine tomographic imaging technique that uses gamma rays. The system shares a number of similarities with conventional nuclear medicine planar imaging using a gamma camera; however, SPECT is capable of providing true 3D information. This information is typically presented as cross-sectional slices through the patient or animal; however, the information can be presented in other ways and can be freely reformatted or manipulated as required. SPECT imaging is performed using a gamma camera to acquire multiple 2D images (projections) from multiple angles. A computer is then used to apply a tomographic reconstruction algorithm to the multiple projections to yield a 3D dataset. This dataset can be manipulated to show thin slices along any chosen axis of the body similar to those obtained using other imagining techniques, such as MRI, CT and PET. To acquire SPECT images, the gamma camera is rotated around the patient and projections (images) are acquired at defined points during the rotation, typically every 3-6 degrees. Conventionally, a full 360 degree rotation is used to obtain an optimal reconstruction. Multi-headed gamma cameras can be used to accelerate the acquisition process. SPECT can be used to complement any gamma imaging study where a true 3D representation can be helpful.

Another imaging technique that is commonly used is called positron emission tomography (PET) which is a nuclear medicine imaging technique which produces a three-dimensional image or map of functional processes in the body. The system detects pairs of gamma rays emitted indirectly by a positron-emitting radionucltide (tracer) which is introduced into the body on a biological active molecule. Images of tracer concentration in 3-dimensional space within the body are then reconstructed by computer analysis. To conduct the scan, a short-lived radioactive tracer isotope, which decays by emitting a positron, which also has been chemically incorporated into a biologically active molecule is injected into the living object—typically into blood circulation. There is a short waiting period while the active molecule becomes concentrated in tissues of interest and then the research subject (animal) or patient is placed in the imaging scanner. As the radioisotope undergoes positron emission decay, it emits a positron (the antimatter counterpart of an electron). The positron travels up to a few millimeters and encounters and annihilates with an electron, thereby producing a pair of gamma photons moving in opposite directions. The photons are detected when they reach a scintillator material in the scanning device and create a burst of light which is detected by photomultiplier tubes or photodiodes. The PET technique depends on simultaneous or coincident detection of the pair of photos and consequently, photons which do not arrive in pairs are ignored. PET scans are increasingly read alongside CT or MRI scans since the combination gives both anatomic and metabolic information. Because the two scans can be performed in immediate sequence during the same session and the subject or patient does not have to change position, the two sets of images are more-precisely registered so that areas of abnormality on the PET imaging can be more perfectly correlated with anatomy on the CT images.

Yet another imaging technique is computed tomography (CT) which is a medical imaging method employing tomography. Digital geometry processing is used to generate a three-dimensional image of the inside of an object from a large series of two-dimensional X-ray images taken around a single-axis of rotation. CT produces a volume of data which can be manipulated through a process that is known as windowing.

It is therefore desirable to construct and provide an apparatus and method that provides a single technology that combines MR, SPECT and PET imaging techniques in studies where the subject or patient is conscious and in particular in awake animal imaging studies.

### SUMMARY

The apparatus and imaging method of the present invention incorporates and permits the use of a single technology to combine MR, SPECT and PET in awake animal imaging studies. The animal's head is held in a MR compatible restrainer and fiducial markers that are both MR and SPECT/PET sensitive are placed on the head. A high resolution anatomical image can then be obtained of the brain. Without changing the position of the animal's head, the same restraining device, namely, the apparatus of the present invention, is placed into a SPECT or PET scanner while the animal is awake. Radiolabelled tracers used to probe the molecular biology of the brain are imaged. Afterwards the two sets of imaging data, MRI and SPECT/PET, can be coregistered with the aid of the fiducials. To make these restraining devices compatible to both imaging modalities, the apparatus has been designed to reduce the mass that would attenuate the radiation energy. Hollow cores, and open spaces help achieve this goal without compromising the structural integrity needed to restrain an animal's head for awake imaging studies. This technology can be designed for any animal.

The apparatus of the present invention also includes an improved manner of securing the head with friction pads and ear bars. The present apparatus includes an open design for easy placement and positioning of the animals in the restrainer. A third coil (neck coil) can be incorporated into the restrainer as a means of ascertaining measures of blood flow to the brain. By combining the neck coil with the other two coils, a three coil system for all animal experiments is provided, thereby permitting additional data to be collected.

These and other aspects, features and advantages shall be apparent from the accompanying Drawings and description of certain embodiments of the invention.

### BRIEF DESCRIPTION OF THE DRAWINGS

FIG. 1 is a side and top perspective view of an assembled multi-coil imaging apparatus, including a restrainer system, according to one embodiment;

FIG. 2 is a side and top perspective view of a multi-coil restrainer system, in an open position;

FIG. 3 is another side and top perspective view of the restrainer system of FIG. 2 in the open position;

FIG. 4 is a side and top perspective view of the restrainer system of FIG. 2 in a closed position;

FIG. 5 is a side and end perspective view of a head holder that is part of the restrainer system of FIG. 2 in a partially opened condition;

FIG. 6 is another side and end perspective view of the head holder of FIG. 5 in a partially opened condition;

FIG. 7 is a side and top perspective view of an assembled multi-coil imaging apparatus, including a restrainer system, according to a second embodiment;

FIG. 8 is a side and top perspective view of the imaging apparatus of FIG. 7 with a volume coil is a predetermined position;

FIG. 9 is a side and top perspective view of a multi-coil restrainer system, in an open position;

FIG. 10 is a side and top perspective view of the restrainer system of FIG. 9 in a closed position;

FIG. 11 is a side and end perspective view of a head holder that is part of the restrainer system of FIG. 10 in a partially opened condition;

FIG. 12 is another side and end perspective view of the head holder of FIG. 11 in a partially opened condition;

FIG. 13 is a side and top perspective view of an assembled multi-coil imaging apparatus, including a restrainer system, according to a third embodiment (without the volume coil being shown);

FIG. 14 is a side and top perspective view of a multi-coil restrainer system, in an open position;

FIG. 15 is a side and top perspective view of a surface coil for use in the system of FIG. 14;

FIG. 16 is a side and top perspective view of a multi-coil restrainer system, in a closed position;

FIG. 17 is a side and top perspective view of a birdcage head holder according to another embodiment;

FIG. 18 is a side and top perspective view of a birdcage head holder of FIG. 17 coupled to a chassis.

### DETAILED DESCRIPTION OF CERTAIN EMBODIMENTS OF THE INVENTION

As described above, SPECT and PET imaging of a target location of interest, such as the brain, provide images of the point source of radiation without any anatomical definition. Consequently, it is necessary to combine these molecular imaging modalities based on radiation decay with CT or MRI to provide anatomical landmarks. MRI is particularly important because unlike CT, it provides high resolution anatomy of soft tissue in the brain.

In accordance with one aspect of the present invention, an apparatus and method are described herein for using a single technology to combine (bundle) a plurality of imaging techniques, including but not limited to MR. SPECT and PET, in an imaging session of a subject or patient, and in particular, in awake animal imaging studies.

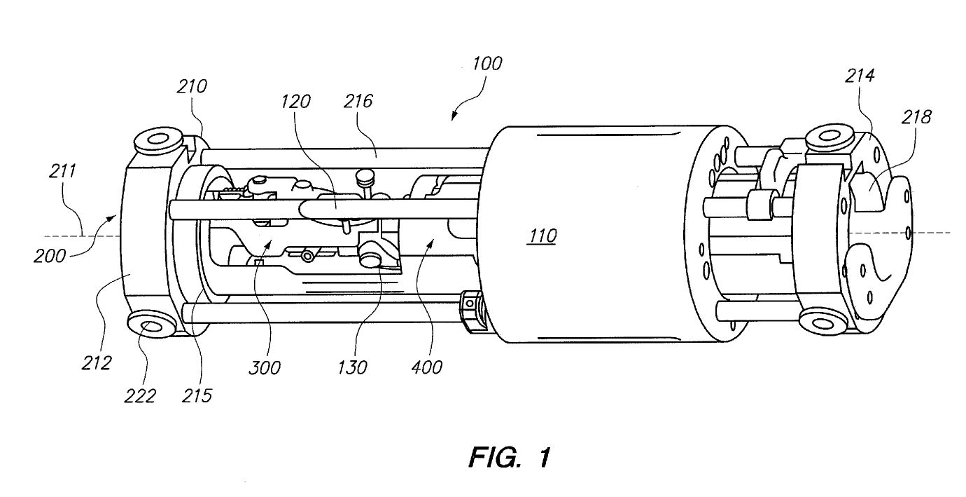

In the figures, like numbers are used to indicate like elements. FIG. 1 shows an assembled multi-component, multi-coil imaging apparatus 100 according to one embodiment of the present invention. The apparatus 100 includes at least two coils as described below and can include three distinct coils, namely, a volume coil 110, a surface coil 120, and a neck coil 130, and is constructed to allow for multimodal imaging of conscious animals that are securely held within an animal restraining assembly 200. The assembly 200 can be used to hold and restrain a number of different animals; however, it is particularly suitable for restraining a rodent, such as a rat, and allow multimodal imaging of the conscious animal. The multi-coil imaging apparatus 100 includes the volume coil 110, which will be described in greater detail below, and the restraining assembly 200 which includes a support frame or chassis 210, a head restrainer or holder 300 and a body restrainer 400.

The frame 210 is shown in perspective in FIG. 1 and has a first or front-end mounting plate 212 and a second or rear-end mounting plate 214 spaced apart by a plurality of support members or rods 216. The front-end mounting plate 212 has a hole (not shown) which is collinear with a longitudinal axis 211 of the frame 210. The rear-end mounting plate 214 also has a cylindrical opening 218 which is collinear with the longitudinal axis 211. The cylindrical opening 218 of rear-end mounting plate 214 is typically larger than the hole on the front-end mounting plate 212. In addition, both the mounting plates 212 and 214 have threaded openings 222 which can receive an adjustable fastening post for centering and securing the frame 210 within a cavity or bore of a piece of equipment, such as a magnetic resonance MR spectrometer.

The support rods 216 position the front-end and rear-end mounting plates 212 and 214 relative to each other and maintain the planes of the plates parallel to each other and perpendicular to the longitudinal axis 211. In addition, the support rods 216 are of such a size and material characteristics that the minor movement of the rear-end mounting plate 214 would not affect movement into the front-end mounting plate 212. In one embodiment, the support rods 216 are connected to the mounting plates 212 and 214 by a damping mechanism, such as rubber gaskets, to further reduce transmission of movement caused on the rear-end mounting plate 214 to propagate to the front-end mounting plate 212. Near the front-end mounting plate 212, a coupling member 215 is formed for coupling the head restrainer 300 and body restrainer 400 to the assembly 200. The coupling member 215 can be in the form of annular shaped collar that receives a portion of the restrainers 300, 400 and provides an attachment surface.

One exemplary frame 210 is described in more detail in commonly assigned U.S. Pat. No. 6,711,430, which is hereby incorporated by reference in its entirety. However, it will be understood that the frame 210 can be constructed in a manner different than the one described in the '430 patent so long as it performs its intended function.

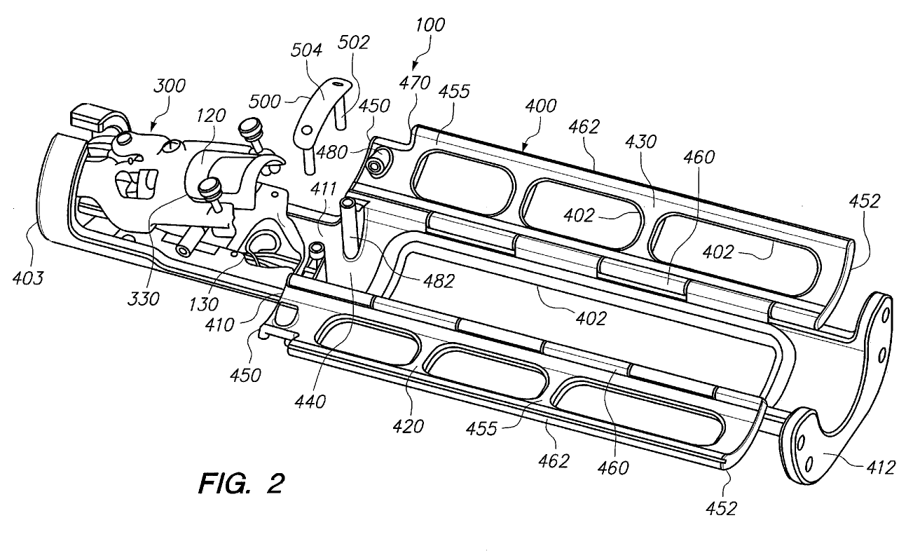

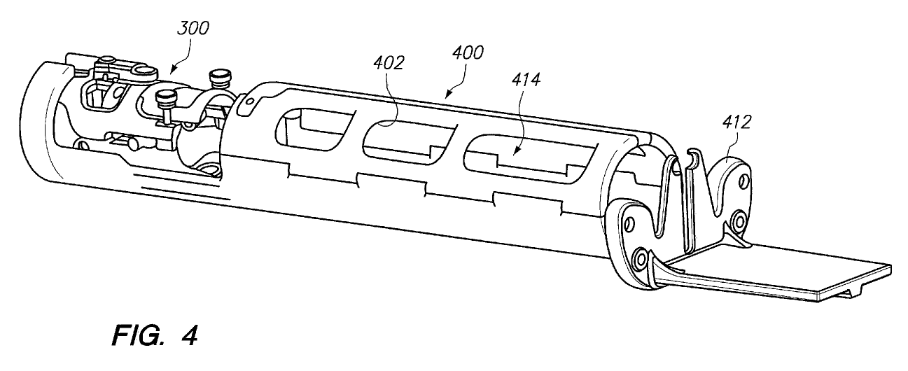

Referring to FIG. 2, the body restrainer 400 has a main body 410 that in the closed position shown in FIG. 4 has a tube-like shape. The body restrainer 400 is formed of a plastic material, such as a Plexiglass material. In contrast to Applicants' previous systems, the body restrainer 400 is a formed as a hollow part and includes the number of openings 402 formed therein along a length thereof. This results in a reduction in mass of the restrainer 400 and provides a more ergomatic part. During the manufacturing process, these openings are formed, thereby reducing not only the mass of the part but Applicants have found the hollow nature of the restrainer 400 and the formation of openings 402 also advantageously reduce the attenuation of the operative signals during the imaging process.

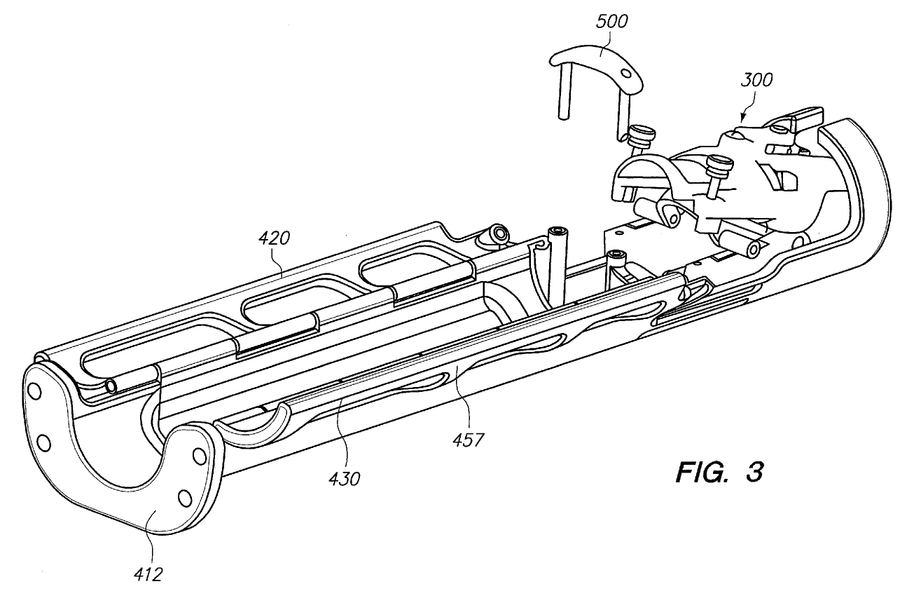

The main body 410 has a flange 412 formed at one end for securing to the rear-end mounting plate 214. The main body 410 also includes first and second wall sections 420, 430 that can be opened and closed relative to one another. For example, FIGS. 2 and 3 show the first and second wall sections 420, 430 open relative to one another and FIG. 4 shows the first and second sections 420, 430 closed relative to one another. The first and second wall sections 420, 430 are coupled to a base section 440 such that the first and second wall sections 420, 430 can move relative thereto. For example, in the embodiment of FIGS. 2-4, the first and second walls sections 420, 430 are hingedly or pivotally coupled to the base section 440; however, it will be appreciated that other means for coupling the sections 420, 430 to the base section 440 can be used. The sections 420, 430 resemble doors that can be opened up in opposite directions to allow access to an interior 414 of the main body where the animal's body is positioned. By allowing the first and second wall sections 420, 430 to open and close, the body of the animal can be more easily inserted into the main body 410 than in previous body restrainers which more resemble a continuous tube.

In the illustrated embodiment, each of first and second sections 420, 430 is in the form of an elongated member (e.g., generally rectangular in shape) that has opposite first and second ends 450, 452 and opposite sides 460, 462. Since the sections 420, 430 and the base section 440 have a generally cylindrical shape in the closed position of FIG. 4, each of these parts has a curvature in that each part is not a planar part but rather has an arcuate shape so that when combined, a generally cylindrical shape is formed. The ends 450 are closer to the head restrainer 300 and the sides 460 are the sides that are coupled to the base section 440 and the sides 462 are the ones that join together in an abutting relationship when the two sections 420, 430 are closed as show in FIG. 4. In FIGS. 2-4, the sections 420, 430 pivot about an axis at one side edge of the base section 440. A previously mentioned, each of the sections 420, 430 has at least one and preferably a plurality of openings 402 formed along a length thereof. As with the rest of the components, such as the base section 440, the sections 420, 430 are hollow parts and the openings 402 can have any number of different shapes including the generally ovoid shape shown in the figures. The area of the openings 402 is a significant amount compared to the total area of the sections 420, 430. Similar to the sections 420, 430, the base section 440 is a hollow member and can include one or more openings 402 to reduce the mass of the part.

The ends 450, 452 can be generally flat, arcuate edges; however, each section 420, 430 includes a cutout 470 near the end 450. A shoulder 472 is formed where the cutout 470 is formed. When the sections 420, 430 are closed, the two cutouts 470 come together to form an opening through which another part (in this case, a shoulder holder or stop 500) can be received as described below. In addition, the sections 420, 430 and 440 have locating and coupling members that assist in securely locating and coupling the shoulder stop 500 in place relative to the other parts. For example, each of the sections 420, 430 includes a hollow first boss member 480 that extends outwardly from an inner surface 455 of each. In the illustrated embodiment, the boss member 480 has a cylindrical shape. The bore that extends through the boss member 480 is open along an outer surface 457 of each section 420, 430. The base section 440 has a pair of complementary coupling members in the form of a pair of upstanding second boss members 482 that extend upwardly and outwardly from a top surface 442 of the base section 440 that defines a floor of the main body 410. The second boss members 482 are also generally cylindrical in shape and are complementary to the first boss members 480 so that when the sections 420, 430 close, the first and second boss members 480, 482 axially align and permit the shoulder holder 500 to be received therein. The bores of the members 480, 482 thus axially align with each other. For example, when the sections 420, 430 are closed, the distal ends of the first boss members 480 seat against distal ends of the second boss members 482. For example, a releasable friction fit can result between the structures. All of the coupling members are preferably integrally formed with their respective sections during the manufacturing process (e.g., molding process).

The shoulder holder 500 is designed to mate with the boss members 480, 482 and can be constructed to have a pair of legs or pins 502 that outwardly from a main body 504 that extends between the pins 502 at proximal ends thereof. The main body 504 thus represents a bar that extends across the pins 502 and since it is designed to seat against the shoulder area of the animal, an inner surface of the main body 504 can be a curved surface to complement the slightly curved nature of the shoulder area. The pins 502 can be formed at right angles to the main body 504. The shoulder holder 500 limits movement of the shoulder of the animal toward the head retainer 300. The shoulder of the animal can be retained between the pair of pins 502 which drop into the bores of the coupling members 480, 482. The choice of the pins 502 is dependent on several factors including the size and type of animal to be restrained.

The main body 410 also has a cutout portion 450, as seen in FIG. 4, which allows access to the head restraining unit (retainer) 300. In addition, there can be a slot and an opening 452 into the cutout portion 450 to prevent coupling of the body retainer 400 with the head restrainer (holder) 300.

The head restrainer 300 includes a first part 310 and a second part 320 that are coupled to one another to allow the parts to be placed in an open position or a closed position. As shown in FIG. 5, the first part 310 can be pivotally attached to the second part 320 to allow pivoting between the two parts. As with the other components, the head restrainer 300 is formed as a hollow plastic part that includes a number of openings 301 to reduce mass and reduce attenuation of the signal. The first part 310 has a first end 312 and an opposing second end 314, with the first end 312 being positioned proximate one end of the body restrainer 400 when the entire unit is assembled. The first part 310 is generally arcuate in shape and can be generally hemi-spherical in shape. The first part 310 includes a slot 316 formed at and open along the first end 312 and a cutout 330 is formed at and is open along the second end 314.

The cutout 330 is formed between and is defined by a pair of arms 340 that are spaced apart from one another. The arms 340 have a top surface 342 and a bottom surface 344. At distal ends of the arms 340 (which also defines the second end 314), a pair of spacers or tabs (protrusions) 346 is formed and extend outwardly and downwardly from the bottom surface 344. In the illustrated embodiment, the tabs 346 have a generally rectangular shape. An inner surface of the tabs 346 can include a guide channel 345 to assist in guiding a fastener 347 that serves to securely attach the first part 310 to the second part 320. In the illustrated embodiment, the fastener 347 is in the form of a pin that includes a head 349. The pin 347 is inserted into an opening 341 that is formed along the top surface 342 of the arm 340 and since the arm 340 is hollow, as the pin 347 travels downwardly toward the second part 330, the guide channel 345 assists in maintaining the pin 347 in the desired orientation and permit it to be more easily received into a complementary opening formed in the second part 320 as described below.

The first part 310 has a bottom edge 350 which is defined substantially by the bottom surface 344 of the arms 340. In accordance with the present invention, the bottom edge 350 includes a member that changes the surface characteristics thereof and in particular, the bottom edges 350 of the arms 340 include first friction pads or strips 370. The strips 370 are elongated structures that run along the length of the arms 340 and are designed to provide a friction, gripping surface.

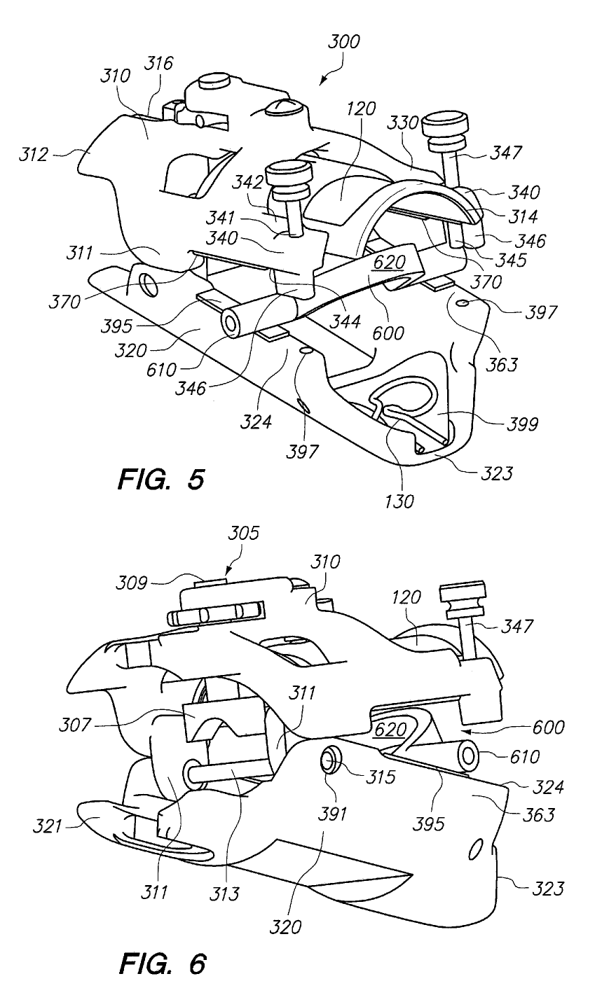

As shown in FIGS. 5 and 6, near the first end 312, the first part 310 has a pair of ear-like structures 311 that extend downwardly and represent the bottommost structure of the first part 310. The ear structures 311 are spaced apart from one another and include an elongated shaft or axle or the like 313 that extends therebetween. In particular, the shaft 313 extends in the inner space between the ear structures 311 and also end portions 315 of the shaft 313 extend beyond the outer surfaces of the ear structures 311. The end portions 315 thus represent stubs that are used to couple the first part 310 to the second part 320 and permit the two parts 310 to open and close relative to one another.

The head restrainer 300 can also include a snout or nose holder or clamp 305 that is adjustable relative to the first part 310. The snout holder 305 has a base portion 307 at a distal end that has an arcute (concave) lower surface to seat against the nose of the animal in an engaged position for restricting movement of the nose when the animal is in place. The snout holder 305 has a shaft that extends from the base portion 307 and terminates in a head 309 at the other end. The shaft passes through a bore formed in the first part 310 and the head 309 is located along the top surface thereof for manipulation by the user (operator). For example, the user can rotate the head 309 to cause an upward or downward movement of the shaft and the base portion 307. When the shaft is rotated in one direction, the base portion 307 is lowered toward the nose of the animal and conversely, when the shaft is rotated in an opposite direction, the base portion 307 is raised away from the nose of the animal. The base portion 307 is formed at least partially between or near the ears 311.

As shown in FIG. 5, the surface coil 120 is disposed between the arms 340. The surface coil 120 has an arcuate shape that is defined by an inner surface 122 that faces the animal and an outer surface 124. One end of the surface coil 120 is disposed underneath the first part 310 while the rest of the surface coil 120 is disposed and accessible between the arms 340. Any number of techniques can be used to securely attach the surface coil 120 to the first part 310 and preferably, the surface coil 120 is detachably attached to the first part 310.

The second part 320 is complementary to the first part 310 and is generally in the form of a cradle in which the animal's head is received and retained during the imaging. The second part 320 has a first end 321 and an opposing second end 323 and includes a pair of upstanding side rails (side walls) 324 that are spaced apart from one another. The second part 320 has a generally arcuate shape and in particular, an inner surface of the second part 320 can be a curved surface (e.g., concave surface) and therefore, when the first and second parts 310, 320 mate together, the inner surfaces thereof define a cylindrical shape to accommodate the animal's round head. As with the other parts, the second part 320 is a hollow plastic part and includes one or more openings 301 that reduce its mass and reduces the attenuation of the signal.

Each of the side rails 324 includes an opening 391 into which one end (end portion 315) of the shaft 313 is received, thereby pivotally attaching the first part 310 to the second part 320. The shaft 313 thus defines a pivot axis about which the first part 310 pivots to allow the first part 310 to open and close relative to the second part 320. The opening 391 can be a through hole that extends completely through the side rail 324. Each side rail 324 includes an elongated top edge 363. The top edge 363 is located so that when the first and second parts 310, 320 are mated together, the top edge 363 faces the bottom edge 350 of the first part 310. At least a portion of each of the top edge 363 and bottom edge 350 is planar. Similar and complementary to the first part 310, the top edge 363 includes a member that changes the surface characteristics thereof and in particular, the top edges 363 of the side rails 324 include second friction pads or strips 395.

The side rail 324 also includes a hole or opening 397 for receiving the pin 347. The opening 397 is located just beyond the second friction pad 395.

The opening 397 and pin 347 can be threaded so as to allow the first and second parts 310, 320 to be securely attached to one another by simply threadingly mating the pin 347 to the side wall of the opening 397.

At the second end 323, the second part 320 includes a recessed compartment or tray 399 that is formed in a floor or bottom portion of the second part 320. The recessed compartment 399 is formed in the bottom portion at a location beyond and adjacent the rails 324. In the illustrated embodiment, the recessed compartment 399 has a triangular shape and is open along the second end 323 of the second part 320. As will be described herein, the neck coil 130 is disposed and held within the recessed compartment 399.

The head restrainer 300 also includes an ear bar 600 that is designed to be attached to the head of the animal. In particular, the ear bar 600 is a separate part that is preferably a hollow structure and includes two axial end sections 610 (aligned axially with one another) and a central arcuate section 620 that is between the end sections 610. The end sections 610 are in the shape of tubes and have a bore 612 that extends therethrough and therefore, each end section 610 can be open at its ends. The actuate section 620 has a semi-circular shape and an inner surface thereof faces the animal's head. The bore 612 is thus open along the inner surface of the arcuate section 620 to allow ear clamps or plugs (not shown) or the like to be inserted therein. These ear clamps are held by the ear bar 600 and are used to position the animal in the head restrainer 300.

The arcuate section 620 is dimensioned so that it is received between the side rails 324, while the ends sections 610 have lengths that allow the end sections 610 to extend beyond the side rails when the ear bar 600 is placed in the space between the first and second parts 310, 320.

The circular shape of the end sections 610 allow the ear bar 600 to freely rotate along the top edge of the side rails 324. In particular, the end sections 610 can be placed on the second friction pads 395 when the head restrainer 300 is in the open position and since the end sections 610 are cylindrical in shape, the heat restrainer 300 can be freely pivoted on the friction pads 395 to place the arcuate section 620 in a desired orientation. It will be appreciated that the end sections 610 can be placed anywhere along the length of the rails 324 (second friction pads 395 thereof) by simply adjusting the location of the end sections 610. The second friction pads 395 engage the end sections 610 and prevent free movement (rotation) thereof.

To securely lock and hold the ear bar 600 in place, the first part 310 is closed relative to the second part 320 causing the first friction pads 370 to come into contact with the end sections 610, thereby resulting in the end sections 610 being effectively nested between the first and second parts 310, 320. By disposing and holding the ear bar 600 between and against opposing friction surfaces 370, 395, the ear bar 600 is held in a desired orientation and in particular, the location and angle of the arcuate section 620 is at least substantially fixed. As mentioned above, in the closed position, the top edges 363 and bottom edges 350 are at least substantially parallel to one another and the first part 310 can be securely attached to the second part 320 using pins 347, with the ear bar 600 being captured therebetween.

The easy adjustment of both the location of the ear bar 600 and the angle of the arcuate section 620 allows for multiple sized animals to be placed in the head restrainer 300 and allows for the variation in animal size that exists within a class of animals, such as rats.

As shown in FIGS. 1-4, the head restrainer 300 is disposed in the cutout 450 of the main body 410 and therefore can be easily inserted and removed from the cutout 450. The head restrainer 300 can be attached to the main body 410 using any number of different techniques. For example, within the cutout 450, the main body 410 includes a floor 411 that can have an arcuate shape (concave) and is defined by two opposing side edges. The side edges are spaced a sufficient distance to allow receipt of the head restrainer 300 and allow the second part 320 to be securely disposed therebetween. The second part 320 can thus be securely attached to the floor of the main body 410 using conventional techniques, such as fasteners, etc. When the head restrainer 300 is disposed in the cutout 450, the first end (ends 312, 321) is disposed near the first end 403 of the main body 410 and the second end (ends 314, 323) is located proximate the ends 450 of the wall sections 420, 430. The head restrainer 300 is securely attached to the main body 410 in such a manner that the parts 310, 320 of the head restrainer 300 can open and close relative to one another without any interference from the main body 410. Similarly, the head restrainer 300 is offset from the wall sections 420, 430 to allow the free opening and closing of the wall sections 420, 430.

It will also be understood that while the wall sections 420, 430 are shown as two separate door-like sections that can pivot, the sections 420, 430 can instead take the form of a single cover that is hingedly attached at one end to the main body 410. In this embodiment, the hinge is located near the flange 412 and the cover thus pivots upward away from the head restrainer 300.

The surface coil 120 which is located within the head restrainer 300, works in conjunction with the volume coil 110 and optionally in conjunction with the neck coil 130 and a system operating controller to produce the image. Details concerning suitable and exemplary imaging devices, such as an MRI device, can be found in the previously incorporated '430 patent.

According to the present invention, the multi-coil apparatus 100 including the restraining assembly 200 and the volume coil 110 and surface coil 120, and optionally the neck coil 130, are installed into the imaging device (e.g., into a tunnel bore thereof). The volume coil 110 is capable of moving along the support rods 216 of the frame 200 to permit easy adjustment thereof and permit the proper positioning of the volume coil 110 relative to the animal's body. Both the surface coil 120 and the volume coil 110 are connected via wiring which extends out of the tunnel bore to a transceiver unit of the system operating controller. In one embodiment, the volume coil 110 transmits and the surface coil 120 is used for receiving. In other embodiments the surface coil both transmits and receives or the volume coil transmits and receives. Similarly, the neck coil 130 can be used to either transmit or receive or both transmit and receive.

Specific details of the construction of each of the coils and the interface between the coils is disclosed in the '430 patent.

Both the TEM volume coil 110 and the surface coil 120 and optionally the neck coil 130 are connected to a transceiver unit. The transceiver unit has an RF transmitter, an RF receiver and a system controller. The system controller controls a pair of switching circuits to transmit and receive the signal from the proper coil 110, 120 or 130. In addition, the system controller also can control an interface to provide active tuning/detuning of the coils. For instance, if the TEM volume coil 110, also referred to as the body coil, is active, transmitting RF energy to the animal, the surface coil 120 is detuned in order to avoid interference. Conversely, when the surface coil 120 is receiving the MR signal from the animal, the TEM volume coil 110 is detuned.

The volume coil 120 can have a cylindrical non-metal core module. The core module has a cylindrical bore that extends through the core module along a longitudinal axis. The cylindrical bore defines an inner surface. In addition, the core module has a plurality of bores extending through the annular core module parallel to and spaced from the longitudinal axis. The apertures accept the support rods 216 to allow the volume coil 110 to move relative to the restraining assembly 200. The volume coil 110 has a plurality of conductive strip lines extending parallel to the longitudinal axis on the inner surface of the core module.

The volume coil 110 has a pair of printed circuit boards (PCB) mounted on the outer, side edges of the core module. In addition, the volume coil 110 has shielding which overlies the core module. The shielding is formed in strips to reduce the occurrence of eddy currents induced by the gradient coils.

The shielding in strips forms a plurality of coaxial slots along the coil's length which serve to interrupt switched gradient induced eddy propagation. Reactively bridged azimuthal slots can extend around the TEM coil's outer wall, end walls, and inner “wall” further limit eddies, and extend the coil's frequency band and dimensional options.

In addition to the shielding being strips, the conductive strip lines creates slots that interrupt eddy current propagation in the TEM coil divide the TEM cavity wall, front to back. The inner elements can be flat, copper foil double-sided strip-line elements, split coaxial elements, or single line copper conductors. This segmented TEM coil combines the internal line element with the external cavity segment, the shielding, forming a resonance circuit. Each functional element can be sub-divided capacitively into one through four or more segments. Trimmer capacitors on the outside wall of the coil are one such division. As in a simple surface coil, the number of capacitive divisions in each resonant unit can be chosen to be few when a more inductive, lower frequency performance of the TEM coil is desired. In contrast, each unit can be divided four or more times to affect the resonance frequency of this slotted TEM volume coil. Thereby electrically modified, the B₁ field generated by this subdivided coil will have improved field linearity and homogeneity.

A printed circuit board has an exposed surface, the surface of which faces away from the core module of the volume coil 110. The inner surface which is covered with and is part of the shielding along with strips of shielding. The printed circuit board has a plurality of components.

A schematic of exemplary circuitry associated with the volume coil is shown in the '430 patent. The volume coil 110 has a plurality of resonating elements which include the strip lines and the shielding. It is recognized that the TEM volume coil 110 can have more or less resonating elements 146 that are connected to detuning/tuning circuits in order to move the resonance frequency of the resonating elements away from the target resonance so as not to interfere with the receiving coil as explained in further detail below. The volume coil 110 in addition has a matching circuit for adjusting the impedance of the resonating element to that of the RF source. An RF source, the transceiver unit and the detuning source, however, are not part of and are located remote from the volume coil 110 and are connected through coaxial cables which extend out of the cavity and connect to the transceiver unit. The volume coil 110 has an RF decoupling circuit that ensures that the DC detuning signal does not interfere with the RF signal path.

Additional exemplary circuitry is disclosed in the '430 patent and can include a matching circuit includes a variable tunable capacitor. The detuning source is connected to the detuning circuit via a filter circuit and the RF decoupling circuit. The filter circuit has a pair of inductors and a capacitor. The filter is for separating the high frequency RF from interfering with the tuning/detuning signal. The RF decoupling circuit has three radio-frequency chokes (RFC) which represent low resistance to the DC current, but high impedance to the RF signal, thereby decoupling both signals from each other. From the detuning circuit which contains a pair of pin diodes, the resonating element is connected.

The volume coil typically has several inputs including the RF source from the RF transmitter of the transceiver unit, the DC source and a ground. The strip lines are each part of a resonating element. The strip lines are represented in the circuit as distributed inductor in the resonating element. The strip lines, as represented by the inductors, can be connected in series to a pair of capacitors. One of the capacitors is a variable, tuneable capacitor. The variable, tuneable capacitors of one of the resonating elements is located on the front PCB and the variable, tuneable capacitors of the adjacent resonating elements are located on the rear PCB; in that there are an even number of resonating elements, the variable, tuneable capacitors are equally located on the front PCB and the rear PCB. The other capacitor for each resonating element is located on the other PCB than that of the variable, tuneable capacitor.

In an alternative embodiment, all the variable, tuneable capacitors of the resonating elements are located on the front PCB. The other capacitor, the capacitor is located on the rear PCB.

The variable, tuneable capacitors can be tuned manually or electronically. The capacitors are each carried on the printed circuit board. One of the sets of the capacitors and a strip line in conjunction with the outer strip shielding form an element which is connected to the detuning circuit.

Each of the detuning circuits has a pair of diodes. In one embodiment, the diodes are pin diodes. The RF decoupling circuit can have a plurality of inductors. One of the detuning circuits and one of the decoupling circuits can be each interposed between one of the resonating elements and the filter circuit. The filter can be connected to the DC source through a resistor. The DC source is used in operating the circuit in conjunction with surface coil as explained below.

The RF source and the matching circuit can be connected to one of the resonating element. The matching circuit includes the variable tuneable capacitor which can be tuned manually.

As indicated above, the volume coil 110 can have shielding located on the outer surface of the core module. The strips of shielding are connected to each other by capacitors located at alternative ends of the strips of shielding. The capacitors are located on the outer surface of the resonating element as part of the shielding. Additional capacitors may be located at the other end of the strips of shielding or alternatively they may be shorted in an effort to reduce the occurrence of eddy currents due to the activation of the gradient coils. The first element shown connected to a detuning circuit.

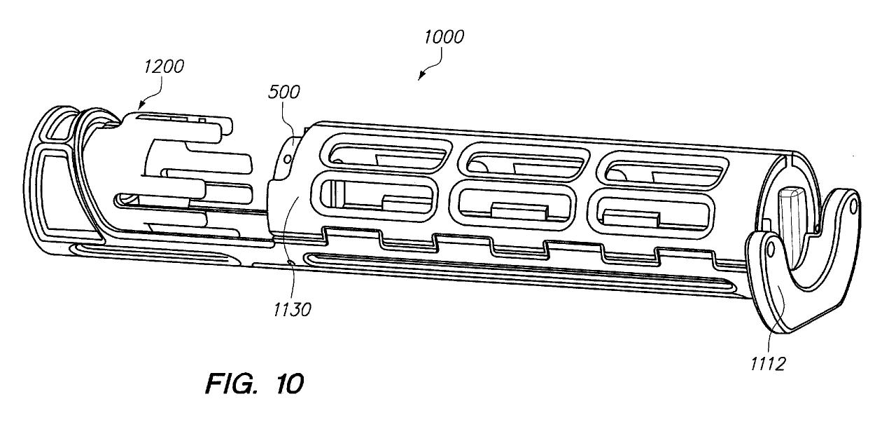

The strips of shielding are connected to each other by capacitors located on the outer surface of the resonating element as part of the shielding such as seen in FIG. 10B. The capacitors are located at alternative ends of the strips of shielding 136. In the embodiment shown, the other end of the strips of shielding 136 are shorted to reduce the occurrence of eddy currents as discussed above.

The surface coil 120 can take various shapes. The surface coil 120 can have a single loop or have multiple loops arranged in a dome shaped surface coil. Exemplary circuitry of the surface coil is disclosed in the '430 patent. The surface coil can have a post or the like for attaching to the head holder (restrainer 300) or other device as explained below. The surface coil can have a pair of connectors which are connected to the RF source and the DC source. Similar to the volume coil 110, the surface coil 120 has a detuning circuit and matching capacitor circuit. Also similar to the volume coil 110, the surface coil 120 can have the inputs of the RF source, the DC source and the ground.

The surface coil 120 can have a plurality of resonating elements, each with a strip line which is represented by an inductor. Both fixed and tuneable capacitors are deployed. The tuneable capacitor is used to adjust the resonance frequency with a capacitor is used to match the circuit.

A method of performing imaging with the apparatus 100 is now described.

The animal can first be placed under light anesthesia before being fitted with the components of the apparatus 100. Both the body restrainer 400 and the head restrainer 300 are prepared for receiving the animal by placing these components in open positions. For example, the wall sections 420, 430 are pivoted to an open position and the first part 310 of the head restrainer 300 is pivoted open. In addition, the shoulder holder 500 is removed to allow the wall sections 420, 430 to pivot open. This general arrangement of parts is shown in FIG. 2.

Next the animal is placed into the body restrainer 400 such that the main torso of the animal is located within the body restrainer 400, with the “shoulder” area of the animal being located near the second boss members 482. The head of the animal is received into the open head restrainer 400 and positioned so that the nose of the animal is disposed below the nose holder 305 which can then be manipulated until the base portion 307 seats against the nose of the animal. The ear holder or bar 600 is attached to the animal's head using fasteners, such as the ear clamps, with the head being received within the concave portion of the arcuate section 620 of the ear holder 600. The arcuate section 620 can be placed in a desired position relative to the head of the animal and then the end sections 610 are disposed in a predetermined position on the second friction surfaces 395. The first part 310 of the head holder 300 is closed causing the first frictions surfaces 370 to engage a top portion of the end sections 610. The first part 310 is then securely attached to the second part 320 using the pins 347, with the ear bar 600 being captured between the first and second parts 310, 320.

Once the animal's head is securely held within the head restrainer below the surface coil 120, the first and second wall sections 420, 430 are closed to form a tube-like structure that surrounds the animal's body. The first and second boss members 480, 482 axially align with one another. Next, the shoulder holder 500 is mated with the main body 410 by inserting the pins 502 into the bores formed by the aligned boss members 480, 482. The insertion of the pins 502 prevents the first and second walls sections 420, 430 from freely opening.

It will be appreciated that the head restrainer 400 can first be securely attached to the head of the animal prior to placement of the animal's body in the body restrainer 400. In this case, the head of the animal is received in the open head restrainer 400 and both the nose holder and ear holder are tightened to securely hold the animal's head in the head restrainer 400.

The head restrainer 300 and body restrainer 400 are coupled to the assembly 200. For example, the head restrainer 300 can be mated with and secured to coupling member 215 of the assembly 200 using conventional techniques. The body restrainer 400 is slipped through the opening 218 of the rear-end mounting plate 214. The volume coil 110 is slid along the support rods 216 to the proper position encircling the animal. The head restrainer 300 is an MR compatible restrainer and therefore, the apparatus 100 can be used in multimodal imaging which includes MR imaging.

The multi-coil apparatus 100 can then be installed into a tunnel bore of the imaging device, such as an MRI device. Before testing, the anesthesia has worn off so that the animal is conscious. The transmit/receive system of the imaging device controls the volume coil 110, surface coil 120 and neck coil 130.

The present invention provides a number of advantages over conventional imaging devices as a result of it being a multi-coil apparatus that provides multi-modal imaging. As previously mentioned, SPECT and PET in the brain provides images of the point source of radiation without any anatomical definition. Hence it is necessary to combine these molecular imaging modalities based on radiation decay with CT or MRI to provide anatomical landmarks. MRI is particularly important because unlike CT is provides high resolution anatomy of soft tissue of the brain. The apparatus 100 of the present invention provides a method for using a single technology to combine MR, SPECT and PET in awake animal imaging studies. The animal's head is held in a MR compatible restrainer 300. Fiducial markers that are both MR and SPECT/PET sensitive are placed on the head.

A high resolution anatomical image is obtained of the brain. Without changing the position of the animal's head, the same restraining device, namely, the apparatus 100 of the present invention, is placed into a SPECT or PET scanner while the animal is awake. Radiolabelled tracers used to probe the molecular biology of the brain are imaged. Afterwards the two sets of imaging data, MRI and SPECT/PET, can be coregistered with the aid of the fiducials. To make these restraining devices compatible to both imaging modalities, the apparatus 100 has been designed to reduce the mass that would attenuate the radiation energy. Hollow cores, and open spaces help achieve this goal without compromising the structural integrity needed to restrain an animal's head for awake imaging studies. This technology can be designed for any animal. The apparatus of the present invention also includes a new way of securing the head with friction pads and ear bars. The present invention includes an open design for easy placement and positioning of the animals in the restrainer. This present invention includes a third coil (neck coil) that provides the means of ascertaining measures of blood flow to the brain. By combining the neck coil with the other two coils, a three coil system for all animal experiments is provided, thereby permitting additional data to be collected.

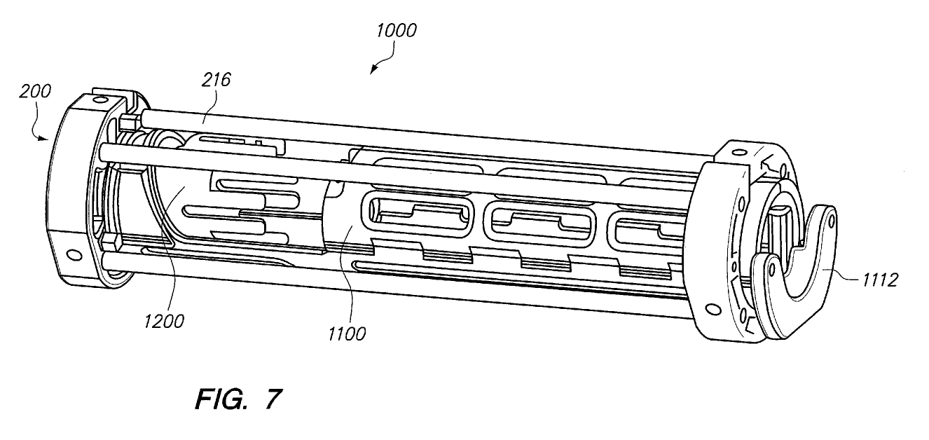

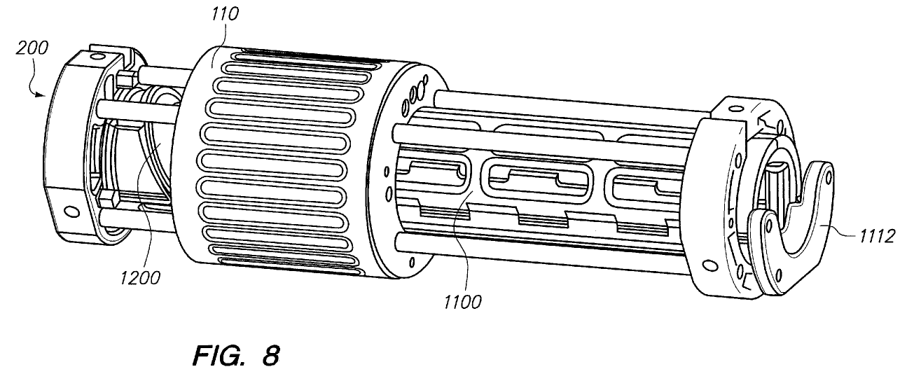

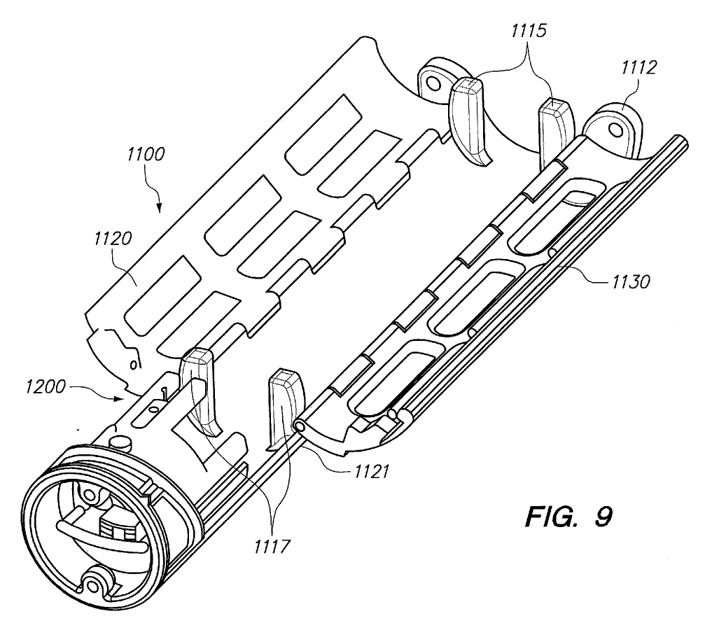

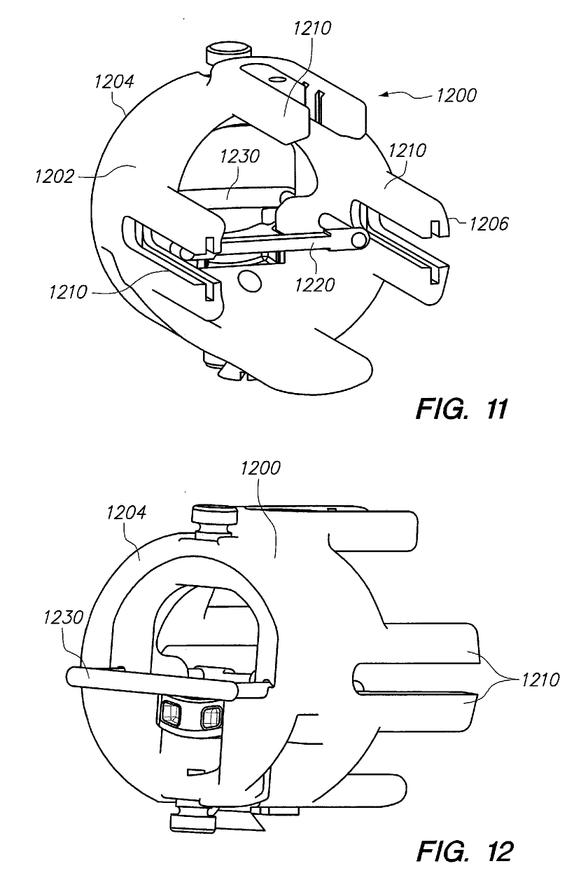

Now referring to FIGS. 7-12 in which a multi-coil imaging apparatus 1000 according to another embodiment of the present invention is shown and is suitable for use with marmosets (small clawed monkeys). As with the previous embodiment, the apparatus 1000 is formed of hollow components that include openings in order to reduce the mass and reduce attenuation of signal.

The apparatus 1000 is similar to the apparatus 100 and therefore like components are numbered alike. For example, the apparatus 1000 can be a two or three coil system the same as the apparatus 100. The volume coil 110 is adjustable along rods 216. A body restrainer 1100 is similar to the body restrainer 400 in that it includes a base section 1110 and a pair of openable and closeable wall sections 1120, 1130. A flange 1112 is formed at one end of the restrainer 1100. The base section 1110 includes a first pair of upstanding posts 1115 and a second pair of upstanding posts 1117. The body restrainer 1100 has a number of openings 1101 formed therein. FIG. 10 shows the wall sections 1120, 1130 in closed positions. As with the first embodiment, instead of having two wall sections 1120, 1130 that are hingedly connected along sides thereof, a single cover can be pivotally attached to the base section 1110.

The body restrainer 1100 also includes a cutout portion 1121 that allows access to a head restrainer 1200. The head restrainer 1200 includes a body 1202 that has a first end 1204 and a second end 1206. The body 1202 includes a number of finger portions 1210 that extend toward and terminate at the second end 1206. The finger portions 1210 include a pair of side fingers that each has a slot 1211 formed therein. The head restrainer 1200 has a chin holder 1220 and a bite bar 1230. When the head restrainer 1200 is inserted into the body restrainer 1100, the first end 1204 is located at the end opposite the flange 1112 and the fingers portions 1210 are disposed within and accessible through the cutout portion 1121.

As with the previous embodiment, any number of different techniques can be used to mount the components of the apparatus 1000. For example, the ends of the body restrainer 1100 can be coupled to the assembly 200. In addition, as with the previous embodiment, the apparatus 1000 has a surface coil that is positioned within or proximate the head restrainer, with the volume coil surrounding the animal and being adjustable along the rods of the frame (chassis).

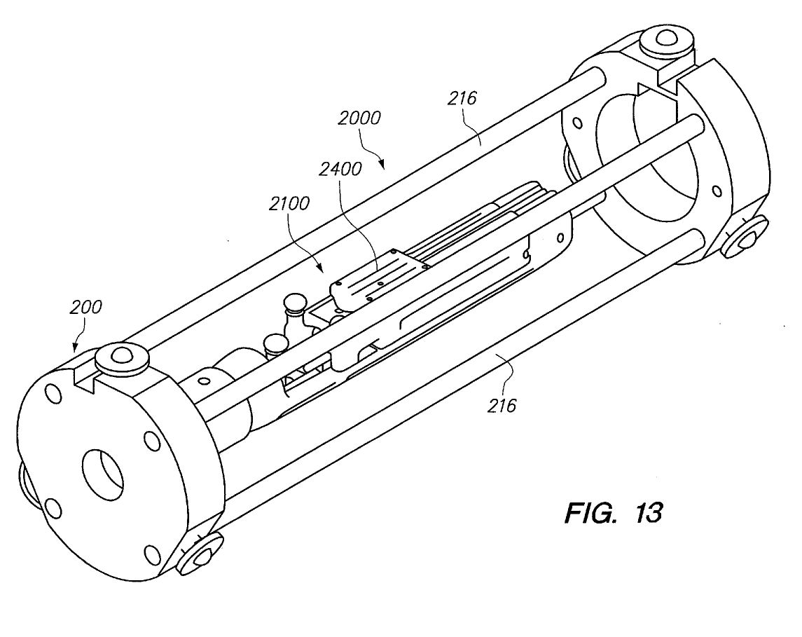

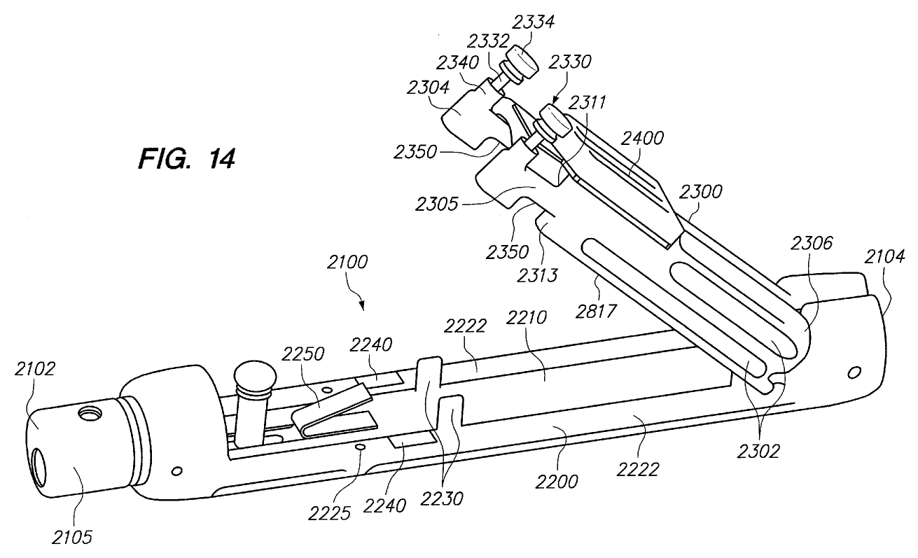

Now referring to FIGS. 13-16 in which a multi-coil imaging apparatus 2000 according to another embodiment of the present invention is shown and is suitable for use with a mouse. As with the previous embodiment, the apparatus 2000 is formed of hollow components that include openings in order to reduce the mass and reduce attenuation of signal.

The apparatus 2000 is similar to the apparatus 100 and therefore like components are numbered alike. For example, the apparatus 2000 can be a two or three coil system the same as the apparatus 100. The volume coil (not shown) is adjustable along rods 216 as described and illustrated in previous embodiments.

The apparatus 2000 includes a body restrainer 2100 that includes a first end 2102 and an opposing second end 2104 and is defined by a first part 2200 and a second part 2300. The first end 2102 can include a coupling member 2105 that is configured to couple the body restrainer 2100 to the assembly 200. The coupling member 2105 illustrated in the figures is generally cylindrical in shape and has openings (slots) and threads to allow coupling between the different parts.

The body restrainer 2100, and in particular, the first part 2200 thereof, has a central compartment 2210 in which the mouse sits. In particular, the central compartment 2210 has a first end and an opposing second end. The central compartment 2210 is defined by a floor section 2220 that has an arcuate shape (semi-circular) and terminates in a pair of opposing side edges 2222. The side edges 2222 are parallel to one another and define planar top surfaces. The floor section 2220 includes a number of features to assist in coupling the first and second parts 2200, 2300 and to assist in positioning and holding the mouse. For example, the first part 2200 can include a pair of shoulder stops 2230 that are upstanding posts that are located opposite one another. The stops 2230 are located within the central compartment 2210 adjacent the side edges 2222 and in fact, the height of the stops 2230 is greater than the heights of the sides of the body restrainer 2100 and therefore, the stops 2230 extend upwardly beyond the top surfaces of the side edges 2222. The stops 2230 can be formed along a length of the central compartment 2210 and in the illustrated embodiment, the stops 2230 are formed at about ½ the distance from one end to the other end of the compartment 2210.

Similar to the body restrainer 400, the body restrainer 2100 has a pair of first friction pads or strips 2240. The strips 2240 are elongated structures that run along a length of the side edges 2222 and are designed to provide a friction, gripping surface. The strips 2240 are located proximate the stops 2230 and are spaced opposite one another. The strips 2240 are thus located between the stops 2230 and the coupling member 2105.

The first part 2200 also includes a tooth bar 2250 that is disposed within the compartment 2210. The tooth bar 2250 is designed to engage the teeth of the mouse when it is securely held and contained within the restrainer 2100. The illustrated tooth bar 2250 is in the form of a V-shaped structure that sits on one of its side and is designed to have some flexing action to accommodate the teeth of the mouse. The open end of the V-shaped structure faces the stops 2230. In addition, the tooth bar 2250 is disposed along the floor section 2220 between the two stops 2230 and therefore is positioned so that when the mouse is disposed between the two stops 2230, the mouth and teeth of the mouse are properly oriented with respect to the tooth bar 2250.

As with the previous embodiments, the second part 2300 is in the form of a convertible cover that is openable and closeable relative to the first part 2200. The second part 2300 like the first part 2200 is a hollow structure that can include openings or slots, such as channels 2302, that reduce the mass of the second part 2300 and reduce attenuation of the signal. The second part 2300 has a first end 2304 and an opposing second end 2306. The second part 2300 is designed as a cover that mates with the first part 2200 and includes a first surface (upper surface) 2310 and an opposing second surface (lower surface) 2320 that faces the first part 2200. The second surface 2320 can have an arcuate contour (e.g., semi-circular shape) to accommodate the shape of the back of the mouse when the parts 2200, 2300 are closed.

The second part 2300 can be coupled to the first part 2200 in any number of different ways, including but not limited to be hingedly coupled, pivotally coupled, etc. In the illustrated embodiment, the second end 2306 of the second part 2300 is coupled to the first part 2200 in a hinged manner to permit the second part 2300 to pivotally open upwards, thereby exposing the inner compartment 2210.

Near the first end 2304, opposing side walls 2305 of the second part 2300 have reduced dimensions and in particularly, a first cutout 2311 is formed along the first surface 2310 and a second cutout 2313 is formed along the second surface 2320. The second cutout 2313 has a U-shape. At the first end 2304, within the first cutout 2311, each side wall 2305 has a coupling member 2330 for coupling and securely attaching the second part 2300 to the first part 2200. The coupling members 2330 can take any number of different forms and in the illustrated embodiment, the coupling members 2330 are in the form of a pair of locking pins defined by an elongated shaft 2332 and a head portion 2334 at one end. The coupling members 2330 are received within upstanding hollow bosses 2340 that are formed at the first end 2304, within the first cutout 2311.

Within each second cutout 2313, a friction pad or strip 2350 is provided and is designed to provide a friction, gripping surface. The strip 2350 is formed generally planar and in a parallel plane compared to the plane of a bottom edge 2317 of the second part 2300. The second part 2300 is coupled to the first part 2200 such that when the second part 2300 is closed as shown in FIG. 16, the second cutouts 2313 are aligned with the first strips 2240 to cause the second strips 2350 to be disposed over the first strips 2240.

Similar to the first embodiment, the restrainer 2100 also includes an ear bar (not shown) that can be similar to ear bar 600 (FIG. 5) but with reduced dimensions compared thereto and is designed to be attached to the head of the animal (mouse). In particular, the ear bar can be a separate part that is preferably a hollow structure and includes two axial end sections (aligned axially with one another) and a central arcuate section that is between the end sections. The end sections are in the shape of tubes and have a bore that extends therethrough and therefore, each end section can be open at its ends. The actuate section has a semi-circular shape and an inner surface thereof faces the animal's head. The bore is thus open along the inner surface of the arcuate section to allow ear clamps or plugs (not shown) or the like to be inserted therein. These ear clamps are held by the ear bar and are used to position the animal in the head restrainer.

The arcuate section can be dimensioned so that it is received between the side edges 2222, while the ends sections have lengths that allow the end sections to extend beyond the side edges 2222 when the ear bar is placed in the space between the first and second parts 2200, 2300.

The circular shape of the end sections allow the ear bar to freely rotate along the top edge of the side edges 2222. In particular, the end sections can be placed on the first friction pads 2240 when the restrainer 2100 is in the open position and since the end sections are cylindrical in shape, the ear bar can be freely pivoted on the friction pads 2240 to place the arcuate section in a desired orientation. It will be appreciated that the end sections can be placed anywhere along the length of the first friction pads 2240 by simply adjusting the location of the end sections. The second friction pads 2350 engage the end sections and prevent free movement (rotation) thereof.

To securely lock and hold the ear bar in place, the second part 2300 is closed relative to the first part 2200 causing the second friction pads 2350 to come into contact with the end sections, thereby resulting in the end sections being effectively nested between the first and second parts 2200, 2300. The underside of the second part 2300 accommodates the stops 2230. The side edges 2222 also have openings 2225 formed therein to receive the coupling members 2330. For example, the shafts 2332 of the pins can be threaded and the openings 2225 can be threaded openings to allow the pins 2330 to threadingly mate therewith resulting in the second part 2300 being securely attached to the first part 2200. As seen in FIG. 16, when the second part 2300 is closed, the tooth bar 2250 is positioned at the first end 2304 of the second part 2300.

As with the other embodiments, the mouse is disposed within the first part 2200 and in particular in the inner compartment 2210 thereof. The shoulder area of the mouse is disposed between the stops 2230 and the mouth of the mouse is positioned at the tooth bar 2250. The head of the mouse is thus outside of the second part 2300.

It will be appreciated that the coupling member 2105 of the restrainer 2100 is coupled to the assembly 200 and in particular can be coupled to coupling part 215. When coupled to the assembly 200, as shown in FIG. 13, the restrainer 2100 is held in a horizontal manner within and between the rods 216.

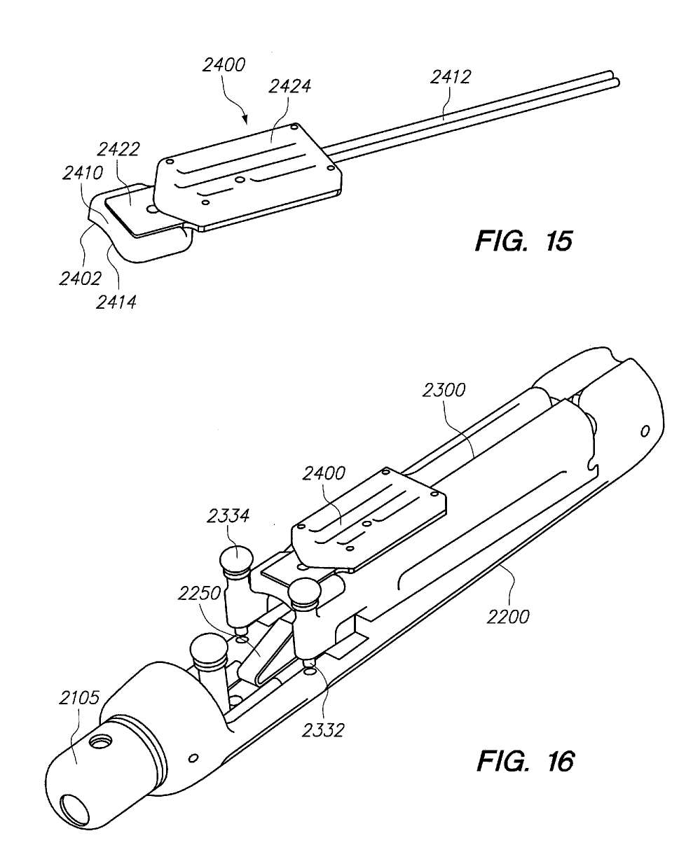

Similar to the other embodiments, a surface coil 2400 is coupled to the restrainer 2100 and in particular, the surface coil 120 for the restrainer 2100 is shown in FIG. 15. The coil 2400 is designed to mate with the second part 2300 and in particular, the coil 2400 is disposed along the top surface of the second part 2300. As shown in FIG. 15, the surface coil 2400 has a base portion 2410 at a first end 2402 of the coil 2400 and a coil portion 2420 that extends from the base portion 2410 and has a pair of wires (contacts) 2412 that extend outwardly therefrom. The base portion 2410 has an arcuate shaped lower surface 2414 (concave) and is designed to be received between and/or within the first cutouts 2311. The coil portion 2420 has a first section 2422 that is coupled to the base portion 2410 and a second section 2424 from which the wires 2412 extend. In use, the surface coil 2400 is attached to the second part 2300 by disposing the base portion 2410 within the space defined by the cutouts 2311 and the second section 2424 lies along the upper surface of the second part 2300. The wires 2412 also run along the upper surface of the second part 2300.

The volume coil (not shown) is disposed about the mouse inside the restrainer 2100 and is positioned near the head thereof, while the surface coil is disposed within the volume coil. The neck coil can be provided in the floor section of the first part 2200.

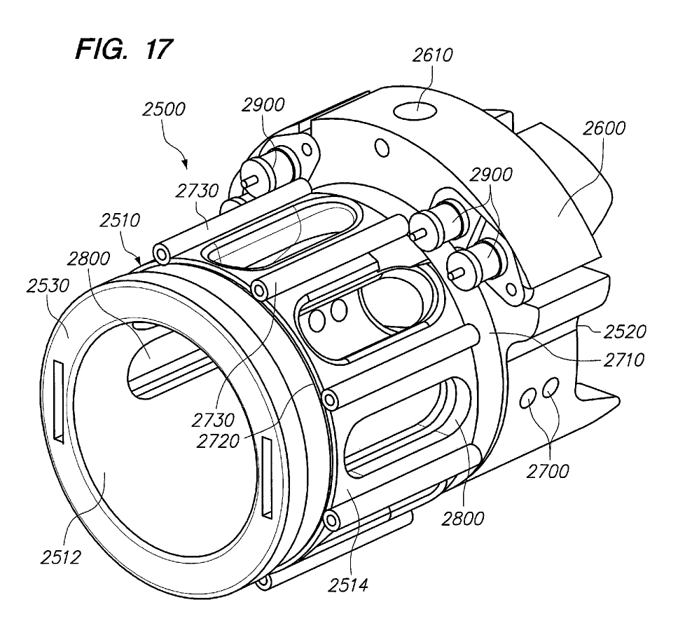

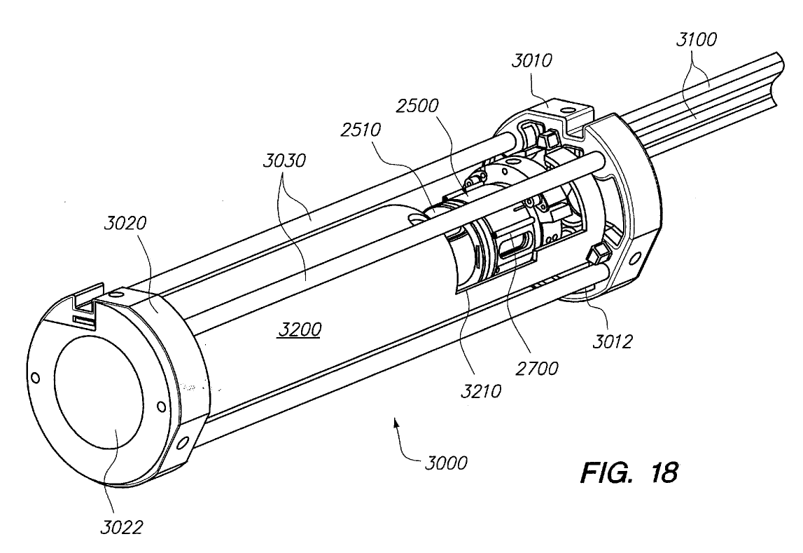

Now turning to FIGS. 17-18 in which a head restrainer or holder 2500 is shown. In this embodiment, the head holder 2500 incorporates imaging components and in particular, the head holder 2500 can be thought of as a birdcage head holder since it incorporates a birdcage coil design. A conventional birdcage coil is an RF coil that is in form of ladder-like resonant networks constructed of solid conductors. More particularly, a conventional birdcage coil is cylindrical in shape and includes two conductive end loops or rings interconnected by an even number of rungs or axial conductors that divide the two end rings into arcs or segments defined therebetween. This construction gives this type of RF coil the appearance of a birdcage and consequently, the term “birdcage” is used to refer to this type of geometry.

The head holder 2500 includes a hollow cylindrical body 2510 that has a first end 2520 and a second end 2530. The body 2510 has a cylindrical shape with both the first end 2520 and the second end 2530 being open. The body 2510 includes an inner surface 2512 and an opposing outer surface 2514.

At or near the first end 2520, the head holder 2500 includes a coil former 2600, such as an acrylic coil former. The former 2600 includes a number of coupling features that permit other components to be coupled thereto. For example and as shown in FIGS. 17 and 18, the former 2600 includes a nose post hole 2610 that is located in a topmost, center portion of the former 2600. As described in Applicant's previous applications, a nose retraining member, such as a nose clamping screw, (not shown) can be inserted into the nose post hole 2610 for engaging and holding the nose of the animal in place. The nose post hole 2610 opens into the open, hollow interior of the body 2510.

In addition, features are provided to permit a bite or tooth bar or the like to be used with the head holder. For example, one or more pair of openings or slots 2700 can be formed near the first end 2520. In the illustrated embodiment, there are two pairs of slots 2700 formed in a lower side portion of the former 2600. It will be appreciated that each pair of slots 2700 is made up of one slot 2700 that is located on one side of the head holder 2500 and another slot 2700 that is formed directly opposite the one slot 2700 in the other side of the head holder 2500. Along one side of the head holder 2500, the slots 2700 are formed linearly to thereby define a plurality of indexed positions within which the bite bar (tooth bar) can be inserted. As described in Applicant's previous applications, an exemplary bite bar includes two end segments that are received within the opposing slots 2700 to thereby position the bite bar across the hollow interior of the head holder. The bite bar extends horizontally along a chord of the circular aperture (hollow interior) of the head holder 2500 to provide a rest for the upper jaw of the restrained animal. The formation of the plurality of pairs of slots 2700 permits the bite bar to have multiple positions to accommodate the scanning of various ROI of the brain by indexing the head of the animal either forwards or backwards. The bite bar is thus positioned within the pair of slot 2700 that positions the head of the animal at a proper location based on the specific body location that the operator is targeting for scanning. For example, if imaging of the frontal lobe of the brain is desired, the bite bar is placed in the pair of slots that are farthest away from the second end 2530 of the head holder.

Other coupling features can be incorporated into the former to allow coupling of the head holder 2500 to other components, etc.

The cylindrical body 2510 of the head holder includes a plurality of slots or openings 2800 that are formed therein and located about the circumference of the body 2510. The illustrated openings 2800 are oblong shaped and extend in the longitudinal direction. It will be appreciated that the openings 2800 not only allow access to the head (e.g., forehead) of the animal but also allows other components to be coupled to the body 2510. For example, lateral ear positioning members, such as the lateral ear clamping screws described in Applicant's previous applications, can be received within the openings 2800. The lateral ear clamping screws are used to position the animal lateral in the head holder 2500. The head holder can also be designed to receive a pair of lower jaw screws for restraining the lower jaw of the animal.

The head holder 2500 includes a coil (birdcage coil) 2700. The illustrated coil 2700 is of a birdcage type and therefore includes a first conducting ring 2710 that is disposed on the outer surface 2514 near or at the first end 2520 of the body 2510 and a second conducting ring 2720 that is spaced from the first conducting ring 2710 and is disposed about the outer surface 2514 near or at the second end 2530 of the body 2510. The birdcage coil 2700 also includes a plurality of rungs 2730 that are electrically connected to the first and second conducting rings 2710, 2720. In the illustrated embodiment, the coil 2700 has eight (8) rungs 2730 with a first rung 2730 being located at approximately 22.5 degrees, such that two of the openings 2800 are located at the 3 and 9 o'clock positions to allow for ear bars or other ear restraining members. In addition, one of the openings 2800 is formed at the 12 o'clock position to accommodate access to the forehead of the animal. The rungs 2730 are disposed and extend longitudinally along the body 2500 in locations between the longitudinal openings 2800.

The coil 2700 is a send/receive type coil and therefore, a separate transmit coil (not shown) is free to be used on other parts of the animal, thereby allowing for multiple portions of the body to be simultaneously imaged. For example, a response to a stimulus can be simultaneously imaged in two unique ROI. Traditionally, a single large coil was used to image multiple locations of the body; however, the quality of the imaging was sacrificed using this traditional approach. For example, brain imaging is obtained using the coil 2700 and a cardiac response to the stimulus can be monitored and imaged using a separate transmit/receive system that is located at a different location of the body.

The coil 2700 also includes a plurality of tuning capacitors which are shown at 2900 that are electrically connected to other coil components and other electronics of the imaging system. In the illustrated embodiment, there are four tuning capacitors 2900 that are generally divided into two pairs.

The frame 3000 has a first or front-end mounting plate 3010 and a second or rear-end mounting plate 3020 spaced apart by a plurality of support members or rods 3030. The front-end mounting plate 3010 has an opening 3012 which is collinear with a longitudinal axis of the frame 3000. The rear-end mounting plate 3020 also has a cylindrical opening 3022 which is collinear with the longitudinal axis. The support rods 3030 position the front-end and rear-end mounting plates 3010, 3020 relative to each other and maintain the planes of the plates parallel to each other and perpendicular to the longitudinal axis. The support rods 3030 can be connected to the mounting plates 3010, 3020 by a damping mechanism.

Additional details about exemplary frames, such as the frame 3000, are set forth in U.S. Pat. No. 6,711,430, which is hereby incorporated by reference in its entirety.

The frame 3000 is also constructed so that tuning rods 3100 can be coupled thereto. Unlike conventional designs, the tuning rods 3100 extend outwardly from the front of the frame 3000 in order to limit the number of components that are contained within the frame 3000 between the ends thereof. More specifically, the front-end mounting plate 3010 includes coupling members, such as short funnel structures, that permit the tuning rods 3100 to be removably coupled to the frame 300.

The removability of the tuning rods 3100 allows for additional animals to be placed in the line of sight of the study animal as stimulation is applied. For example, when the animal is fully restrained within the system, the stimuli are often positioned at the end of the frame 3000 near the front-end mounting plate 3010 and therefore, removal of the tuning rods 3100 provides a number of advantages in terms of positioning the stimuli, accessing the animal, and as discussed above, allowing additional animals to be placed in the line of sight. For example, in certain studies, a predator of the animal restrained in the imaging system acts as the stimulation and therefore, the predator is placed near the front-end mounting plate 3010 to permit the restrained animal to see its natural predator.

FIG. 18 also shows a body restrainer 3200 that is coupled to the head holder and is designed to restrain the body of the animal. Details of exemplary body restrainer are discussed in Applicant's previous applications and patents. It the illustrated embodiment, the body restrainer 3200 includes a slot or opening 3210 where the head holder is located and permit access to the head holder and thus, access to the head of the animal.

While the invention has been described in connection with certain embodiments thereof, the invention is capable of being practiced in other forms and using other materials and structures. Accordingly, the invention is defined by the recitations in the claims appended hereto and equivalents thereof.

What is claimed is:

1. A restraining assembly for an awake animal that permits multi-modal imaging to be performed within a single imaging device comprising:
a head restrainer that restrains the head of an awake animal;
a body restrainer that holds a body of the awake animal;
a support frame positioned within a cavity of the imaging device, the support frame carrying the head restrainer and the body restrainer;
a volume coil mounted on the support frame, the volume coil extending around the body restrainer; and
an RF surface coil and a third coil associated with the head restrainer for placement proximate the animal's head.

2. The restraining assembly of claim 1, wherein the surface coil is located along a top of the head restrainer and the third coil is located a floor of the head restrainer.

3. The restraining assembly of claim 1, wherein the third coil comprises a neck coil and is configured to provide a means for ascertaining measures of blood flow to the brain.

4. The restraining assembly of claim 1, wherein both the surface coil and the third coil are disposed within the volume coil that surrounds the head restrainer.

5. The restraining assembly of claim 1, wherein the volume coil can move longitudinally along guides of the support frame.

6. The restraining assembly of claim 1, wherein the body restrainer has a main portion that surrounds a torso of the awake animal, the body restrainer includes at least a first part and a second part that can be opened and closed relative to the first part to allow for insertion of the animal into the body restrainer in the open position.

7. The restraining assembly of claim 6, wherein the first part is a base section and the second part is hingedly connected to the first part.

8. The restraining assembly of claim 6, wherein the first part is a base section and the body restrainer includes a third part, the second and third parts being pivotally attached to the base section and configured to open in opposite directions to allow insertion of the animal into the body restrainer.

9. The restraining assembly of claim 8, wherein each of the base section, the second part, and the third part has an arcuate shape such that in the closed position, the main portion has a generally cylindrical shape.

10. The restraining assembly of claim 8, wherein the base section includes a first pair of hollow coupling members and each of the second and third parts includes a second hollow coupling member, wherein in the closed position, the second coupling members axially align with the first coupling members, the assembly including a shoulder holder that includes a pair of pins that are inserted into the hollow coupling members, thereby preventing the second and third parts from being opened.

11. The restraining assembly of claim 10, wherein the shoulder holder has a transverse body portion that extends between the pins and includes a lower surface for seating against the body of the animal.

12. The restraining assembly of claim 10, wherein the coupling members are hollow boss members.

13. The restraining assembly of claim 1, wherein the head holder comprises a first part and a second part, the first part being pivotally coupled to the second part to allow the head holder to have an open position and a closed position, the surface coil being coupled to the first part, while the third coil is coupled to the second part.

14. The restraining assembly of claim 1, further including:
an ear holder that includes a pair of end sections and is configured to position the animal in the head restrainer, wherein the head restrainer includes a first friction pad and a second friction pad spaced from the first friction pad, the end sections being held in intimate contact with and between the first and second friction pads when the head restrainer is in a closed position and conversely, when the head restrainer is in an open position, the ear holder is freely adjustable and removable from the head restrainer.

15. The restraining assembly of claim 14, wherein the ear holder includes an arcuate center portion between the end sections for receiving the head of the animal.

16. The restraining assembly of claim 14, wherein the first friction pad is part of a first part of the head restrainer and the second friction pad is part of a second part of the head restrainer, the first part being pivotally connected to the first part to permit opening and closing of the head restrainer.

17. The restraining assembly of claim 1, wherein the imaging device at least one of MR, SPECT, CT, and PET.

18. A method of conducting multi-modal neuroimaging on an awake animal comprising:
restraining an un-anesthetized animal in a restraining device;
performing a first imaging procedure on a target body part of the unanesthetized animal;
subsequently, placing the restraining device in a second restraining device that is separate from the first imaging device, wherein the animal maintains at least substantially the same position in the restraining device in both the first and second imaging devices; and
performing a second imagining procedure on the target body part.

19. The method of claim 18, wherein the first imaging procedure comprises a functional MRI (fMRI) imaging procedure.

20. The method of claim 18, wherein the target body part comprises a brain of the unanesthetized animal.

21. The method of claim 18, wherein the second imaging procedure comprises a SPECT or PET scan procedure.

22. The method of claim 18, wherein the first restraining device comprises a head holder and body restrainer, each of which has a hollow core construction and includes purposely positioned openings that not only reduce the mass of the head holder and body restrainer but also reduce attenuation of the operative signals during the imaging process.

23. The method of claim 22, wherein the head restrainer includes a first part and a second part pivotable coupled to the first part, wherein the first part carries a first coil and the second part carries a second coil, the head restrainer being configured such that the head of the animal is held in place between the first and second parts.

24. The method of claim 23, wherein the second coil comprises a neck coil and the device further includes a volume coil.

25. The method of claim 24, wherein when one of the volume coil and the surface coil is active, the other coil is detuned.

26. The method of claim 18, further including: a volume coil, a surface coil and a third coil, wherein each of the first and second imaging devices includes a transmit/receive system that controls each of the volume coil, surface coil and third coil.

27. The method of claim 18, further comprising the steps of:
placing fiducial markers on a head of the animal, the markers being both MR and SPECT/PET sensitive; and
after generating first imaging data from the first imaging device and second imaging data from the second imaging device, coregistrating the first and second imaging data with the aid of the fiducial markers.

28. A system for multi-modal imaging of an awake animal comprising:
a first imaging device for performing a first type of imaging;
a second imaging device for performing a second type of imaging that is different from the first type; and
a non-magnetic restrainer assembly for holding a body and head of the animal, wherein the restrainer assembly is constructed for use in both the first and second imaging devices without requiring adjustment of the body of the awake animal.

29. The system of claim 28, wherein the first imaging device comprises a magnetic resonance imaging (MRI) device and the second imaging device comprises a SPECT/PET imaging device, the restrainer assembly being configured to be slidably received and mounted within a bore of the MRI device and received within the SPECT/PET device.
